(19) United States
(12) Patent Application Publication
(10) Pub. No.: US 2021/0236011 A1
(43) Pub. Date: Aug. 5, 2021

(54) METHOD AND APPARATUS FOR ESTIMATING BLOOD PRESSURE

(71) Applicant: Oxford University Innovation Limited, Oxford (GB)

(72) Inventors: Lionel TARASSENKO, Oxford (Oxfordshire) (GB); Mauricio Christian Villarroel MONTOYA, Oxford (Oxfordshire) (GB); Stephen PAYNE, Oxford (Oxfordshire) (GB)

(21) Appl. No.: 17/048,868

(22) PCT Filed: Apr. 17, 2019
(86) PCT No.: PCT/GB2019/051094

§ 371 Date: Oct. 19, 2020

(30) Foreign Application Priority Data
Apr. 20, 2018 (GB) 1806469.1

(51) Int. Cl. A61B 5/021; A61B 5/024; A61B 5/00

(52) U.S. Cl. CPC A61B 5/02125; A61B 5/02416; A61B 5/6898; A61B 5/0077; A61B 5/6803

(57) ABSTRACT

A method and apparatus for measuring blood pressure by measuring a photoplethysmographic (PPG) signal of a user with the arm in a raised position and in a lowered position and measuring the difference in timing between them, which represents a change in pulse transit time. The PPG signal is measured in the wrist of the user relative to the PPG signal in the finger of the user. A camera built into a mobile telephone may form a first optical sensor for measuring the PPG signal in the finger and an attached accessory camera, such as an infrared camera, or an optical sensor in a wrist-worn device to obtain the PPG signal in the wrist. Alternatively, a head-worn device may be used as a second optical sensor. Signal averaging based on the timing of the finger-originating PPG signal is used to average the waveforms in the wrist-originating PPG signal for arm-up and arm-down, and the timing difference is measured between the arm-up averaged waveform and arm-down averaged waveform. A calibration process is used to derive a relationship between the change in pulse transit time and the subjects blood pressure allowing a display of an estimate of the blood pressure of the subject on the screen of the mobile telephone.

The present invention relates to a method and apparatus for measuring a subject's blood pressure in a relatively convenient and non-invasive manner.

The monitoring of vital signs, including blood pressure, has long been an important part of healthcare. While traditional relatively-invasive and labour-intensive ways of measuring vital signs are acceptable in a clinical environment, increasingly the public is interested in monitoring their own vital signs for well-being or long-term health reasons and less-invasive, more convenient ways of monitoring vital signs are required. Blood pressure is one of the most important vital signs and it has been traditionally measured using an inflatable cuff positioned around the subject's upper arm which is inflated to occlude blood flow, and then gradually deflated while monitoring the restoration of blood flow. Automatically-operating cuffs are widely-available for use in clinical environments and at home. Such devices are, however, bulky, intrusive and somewhat inconvenient to use.

Improvements in technology, and in particular the high-quality cameras present in modern devices such as mobile telephones (smartphones), tablet computers, wearable devices etc., together with the available processing power in such devices, has enabled the development of convenient contactless vital signs monitoring. Several vital signs such as heart rate, breathing rate and oxygen saturation can be estimated by using such a camera to detect the photoplethysmographic (PPG) signal, which is a variation in the reflectance of skin at certain wavelengths of light as the volume of blood in the skin capillaries varies in synchrony with the cardiac cycle. This PPG signal can be detected in a standard monochrome or colour video image of the skin taken with a normal video camera such as webcam in a mobile telephone or computing device, known as remote PPG imaging (rPPG or PPGi). WO-A2-2013/027027, for example, discloses estimating the heart rate and breathing rate from a photoplethysmographic image signal. Apps (executable software applications intended to run on a smartphone, tablet device or personal computer) are also widely available which allow a device to estimate heart rate by requiring the user to place a finger in contact with the camera in the device, using the light source provided for the camera's flash to illuminate the finger, and using the camera as a simple optical reflectance detector to detect the variations in reflectance constituting the PPG signal.

While effective techniques for estimating heart rate and breathing rate accurately from the PPG signal have been demonstrated, attempt to measure blood pressure have not been so successful. The paper “Towards ubiquitous blood pressure monitoring via pulse transit time: theory and practice” by Mukkamala et al. (IEEE Trans. Biomed. Eng. 2015 August; 62(8): 1879-1901) discusses the various known ways of measuring blood pressure including catheterisation, auscultation (cuff-based blood vessel occlusion as above), oscillometry, volume clamping and tonometry. It also discusses the relationship between pulse transit time and blood pressure. Pulse transit time (PTT) is the time delay for the pressure wave caused by the heart beating to travel between two arterial sites. This pressure wave can be visualised as a varying acute dilation of the walls of blood vessels propagated across the arterial branches as they carry blood away from the heart to the capillaries. PTT is inversely related to blood pressure such that the pulse transit time reduces (the pressure wave moves more quickly) if the blood pressure is higher. PTT can be measured by measuring the relative timing or phase difference between waveforms indicative of the arterial pulse at different locations in the circulatory system. Consequently if the relationship between PTT and blood pressure is known, measuring the pulse transit time offers a way of measuring arterial blood pressure. The relationship between pulse transit time and blood pressure varies from person to person because it is dependent upon many factors, including, for example, arterial wall stiffness and the viscosity of blood. However, the relationship is relatively constant in an individual subject so once the relationship has been found for a subject, for example by a calibration process involving measuring pulse transit times at a variety of blood pressures measured by one of the conventional methods, that relationship can be used to calculate reasonable estimates of blood pressure from future measurements of pulse transit time in the individual subject.

WO-A1-2016/0977708 discloses a method and apparatus for measuring a haemodynamic parameter, including the pulse transit time, by using a mobile device webcam to obtain a remote PPG signal from an area of skin of a patient and, in particular, to measure the relative timing or phase of the PPG signal in different small regions of interest across the exposed skin of a subject. This can give an indication of the speed of a pulse wave across the skin of a subject and thus an indication of pulse transit time. However a simpler method of measuring PTT would be advantageous.

According to the present invention there is provided a method of estimating blood pressure comprising detecting a first PPG signal at a first location on a subject's body using a first optical sensor of a first device held by the subject's hand; detecting a second PPG signal at a second location on the subject's body using a second optical sensor of a second device; measuring the relative timings of the detected PPG signals and calculating from the relative timings an estimate of the blood pressure. Thus the present invention uses two optical sensors to detect the PPG signal at two different locations on the subject's body and by measuring the relative timing, or phase difference, between the two PPG signals, which is a measure of PTT, it calculates an estimate of the blood pressure. This calculation may be made using a relationship between pulse transit time and the subject's blood pressure obtained in a calibration process.

The invention may further comprise detecting the first and second PPG signals with the subject's arm in a first position and measuring a first relative timing between the detected PPG signals, detecting the first and second PPG signals with the subject's arm in a second position, different from the first position, and measuring a second relative timing between the detected PPG signals, and calculating from the first and second relative timings an estimate of the subject's blood pressure. The first position may be with the arm raised higher than in the second position so that the effect of gravity on the pulse wave is different between the two positions. By measuring the pulse transit times with the arm in two positions, and by including the effect of gravity on the speed of the pulse wave, a better estimate of the blood pressure can be calculated as the measurement in two positions with the differing effects of gravity allow a reduction in the number of variables in the relationship between pulse transit time and blood pressure.

The first location may be on the hand of the subject, for example on the finger and the second location may be on the wrist of the subject (on the same arm), or on the arm, or may be a second location remote from the hand, for example on the head of the subject or on or adjacent an ear of the subject.

Preferably at least one of the optical sensors is placed in contact with the subject's skin to detect the PPG signal. For example, this may involve the subject placing their finger against the first optical sensor. In this embodiment, therefore, the invention is not relying on the remotely obtained PPG image signal. Thus at least one of the optical sensors may be an optical reflectance measuring device, which may comprise a light source for emitting light towards the subject's skin and a light detector for detecting light emitted from the subject's skin.

The first device may be a mobile computing device incorporating a camera, such as a smartphone, tablet computer, or wearable device, and the camera may constitute the first optical sensor. The second device may be an accessory camera which can be in data communication with the first device, and optionally can be physically attached to the first device. In this case the measurement may be made by the subject placing their finger against the one of the cameras (for example the camera built into the mobile computing device), and arranging for their wrist to be close to or in contact with the accessory camera. Alternatively the second device could be a wearable device such as a wrist-worn device (e.g. wristwatch) which includes an optical reflectance sensor incorporating a light source and light detector, an earphone incorporating an optical detector, or could be a head-worn device such an audio headphone, virtual reality (VR) headset or spectacles, again including an optical reflectance sensor.

Preferably the method includes the step of either synchronising the clocks of the two devices to facilitate measurement of the relative timings of the PPG waveforms, or alternatively acquiring the relative timings of the clocks. Thus if the relative timings of the two clocks is known, it is not necessarily that the clocks themselves are synchronised.

The method may comprise the step of signal averaging to improve the signal-to-noise ratio of the PPG signals. Signal averaging involves averaging together a plurality of individual PPG waveforms of the PPG signal to obtain an averaged PPG waveform for that signal. Signal averaging improves the signal-to-noise ratio because, unlike the regular PPG signal, noise is randomly distributed. Preferably the method includes the step of comparing the signal-to-noise ratio of the first and second PPG signals, selecting the PPG signal with the highest signal-to-noise ratio and detecting its amplitude maxima or minima, and using the timings of the detected maxima or minima as reference timings for the signal averaging process on the other of the first and second PPG signals. Thus the cleaner of the two signals is used as the trigger for the signal averaging process.

Preferably the first and second devices are connected together for data communication and the first device may control the second device and perform the measurement of relative timings. The first device may also calculate the estimate of blood pressure from the measured timings. This utilises the excellent computing power available in modern mobile computing devices. Thus the method may be executed by an app running on a smartphone or similar device, the app controlling the smartphone and also the second device.

An alternative aspect of the invention provides a method of calculating an estimate of blood pressure from a measurement of pulse transit time comprising the steps of: measuring a first pulse transit time in a subject's arm with the subject's arm in a first position; measuring a second pulse transit time in a subject's arm with the subject's arm in a second position, the second position being different from the first position, and measuring the difference between the first and second pulse transit times, and calculating from the difference in first and second pulse transit times an estimate of the subject's blood pressure. The first position may be with the arm raised higher than in the second position. For example the first position may be with the arm raised approximately vertically above the shoulder and the second position may be with the arm hanging approximately vertically downwards from the shoulder. This aspect of the invention is applicable to any PTT-based blood pressure measurement method and it utilises the known effects of gravity on the speed of the pulse pressure wave to facilitate calculation of blood pressure from pulse transit time.

Another aspect of the invention provides an apparatus for estimating blood pressure comprising: a first device comprising a first optical sensor for detecting a first PPG signal at a first location on a subject's body, the first device being a handheld device; a second device comprising a second optical sensor for detecting a second PPG signal at a second location on the subject's body; and a programmable data processor configured to control the first and second devices to execute the method of estimating blood pressure according to the invention as discussed above.

As discussed above, the first device may be a mobile computing device incorporating a camera, such as a smartphone, tablet computer, or wearable device, with the first optical sensor being its incorporated camera, and the programmable data processor is the data processor of the mobile computing device. The second device may be an accessory camera which can be in data communication with the first device, and optionally can be physically attached to the first device. Alternatively the second device may be a wrist-worn device, head-worn device such as audio headphone, VR headset or spectacles as discussed above.

The invention may be embodied as an app (executable software application) running on a mobile computing/communication device such as a smartphone, and thus the invention extends to such an executable software application in downloadable form, or when downloaded, or when stored on a non-transitory storage medium.

The invention will be further described by way of non-limitative example with reference to the accompanying drawings in which:

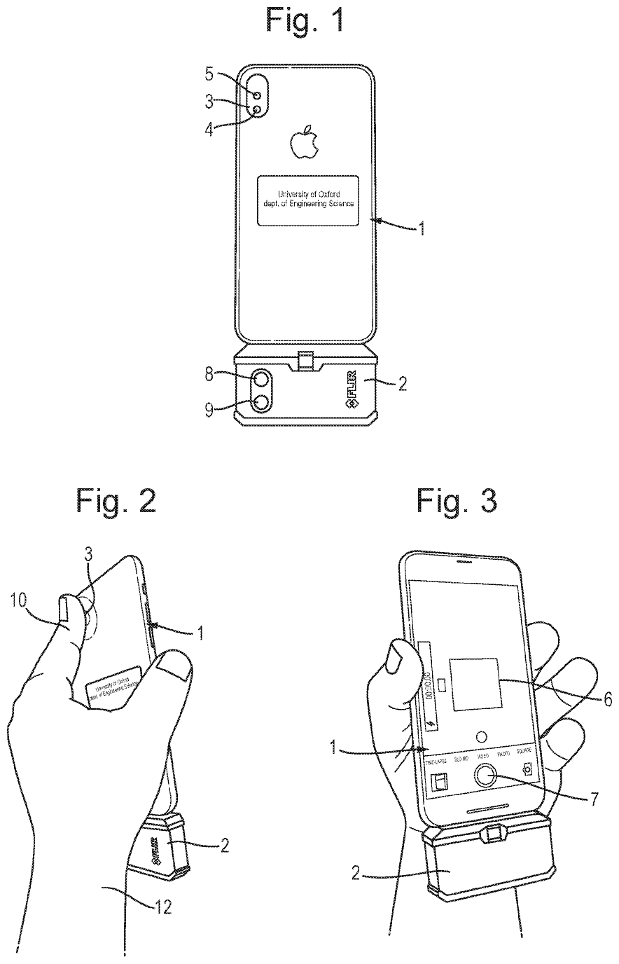

FIG. 1 illustrates a first embodiment of the invention;

FIG. 2 illustrates the embodiment of FIG. 1 in use;

FIG. 3 illustrates the embodiment of FIG. 1 in use;

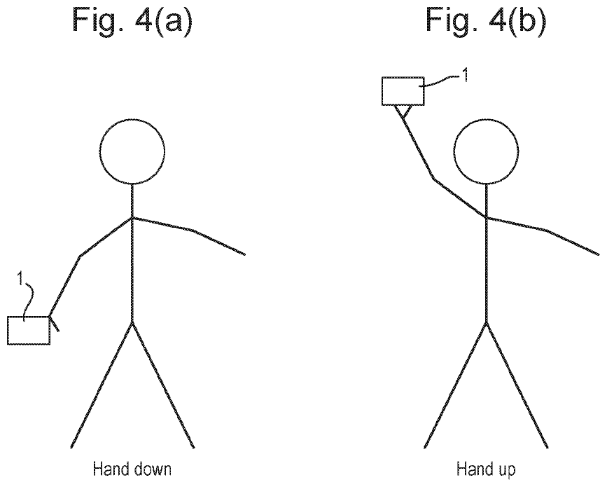

FIGS. 4A and B schematically illustrate the embodiment of FIG. 1 in use;

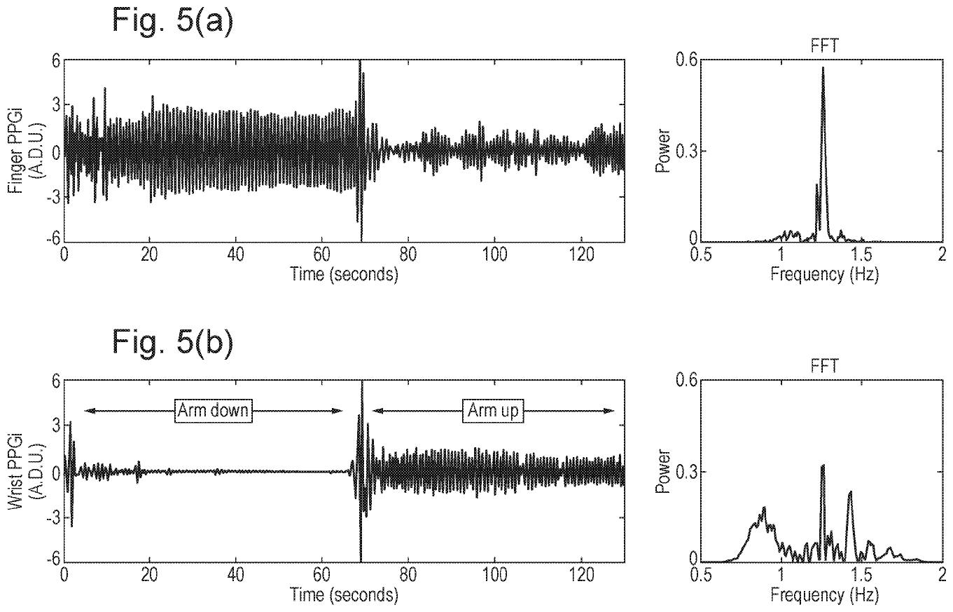

FIG. 5 is a plot of signals obtained with the embodiment of FIG. 1;

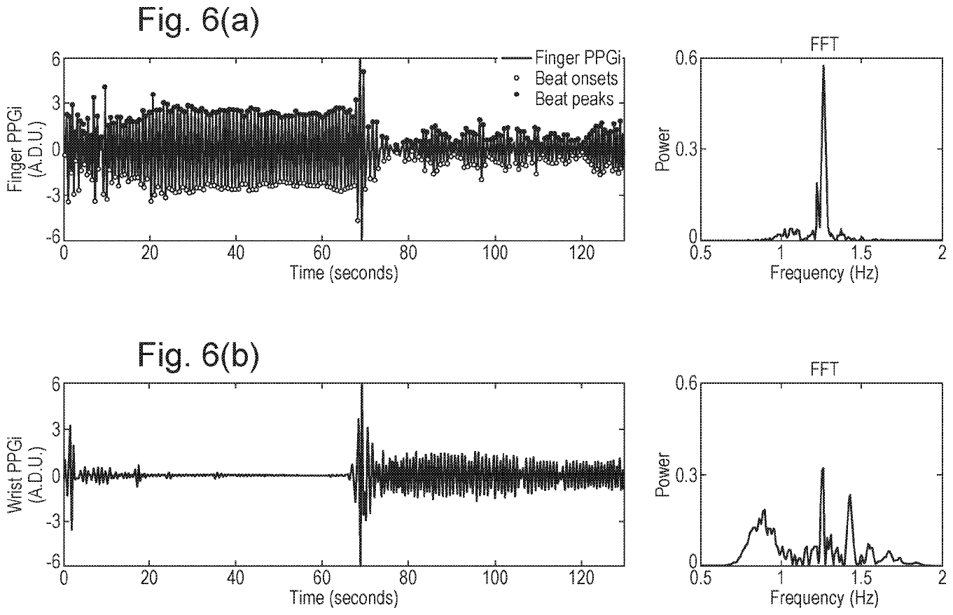

FIG. 6 is a plot of processed signals from the embodiment of FIG. 1;

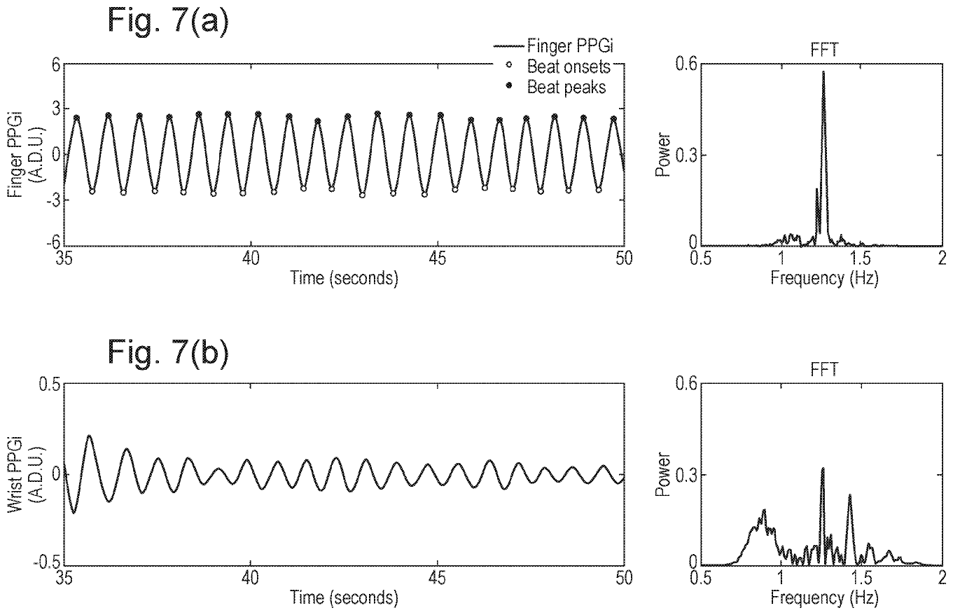

FIG. 7 is an expanded time scale plot of the signals of FIG. 6 (first half—Arm down);

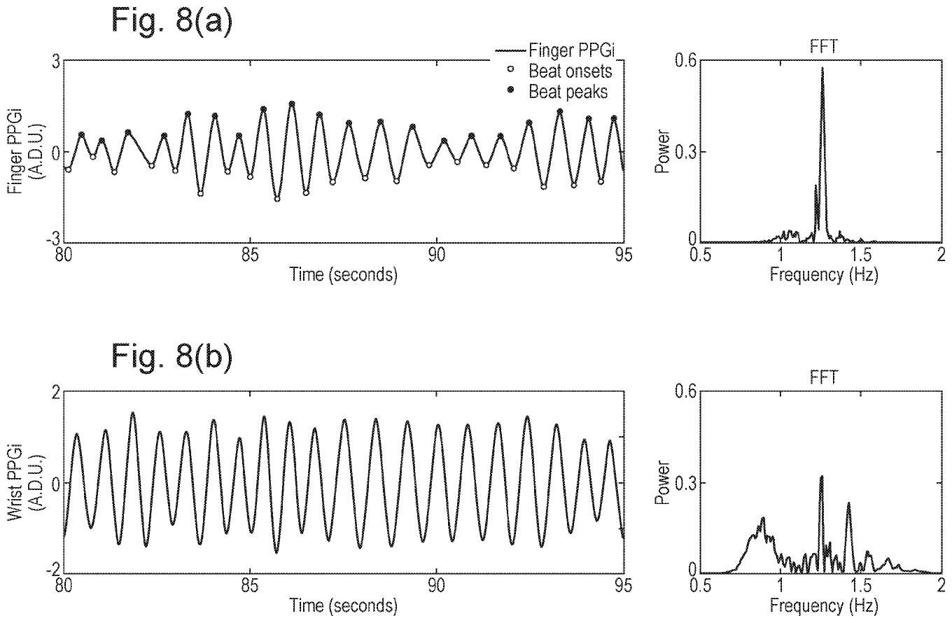

FIG. 8 is an expanded time scale plot of the signals of FIG. 6 (second half—Arm up);

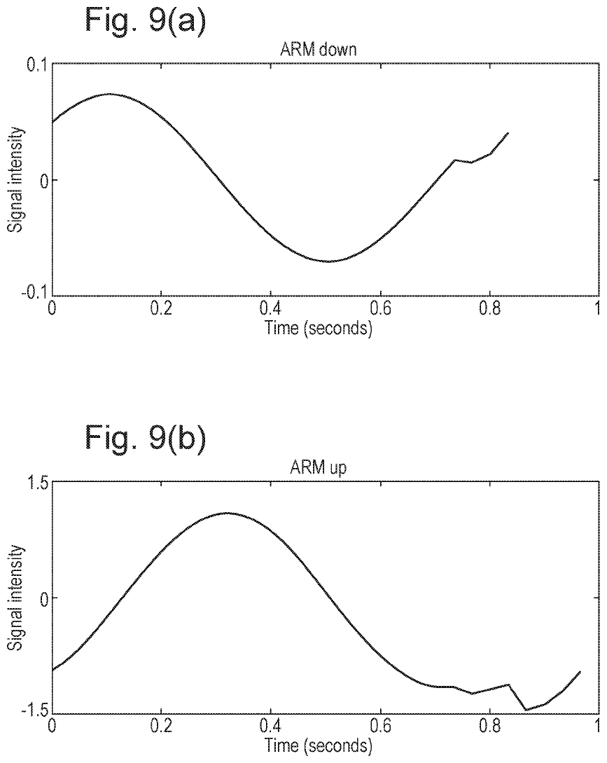

FIGS. 9A and B show signal averaged waveforms of the signals from FIGS. 7 and 8;

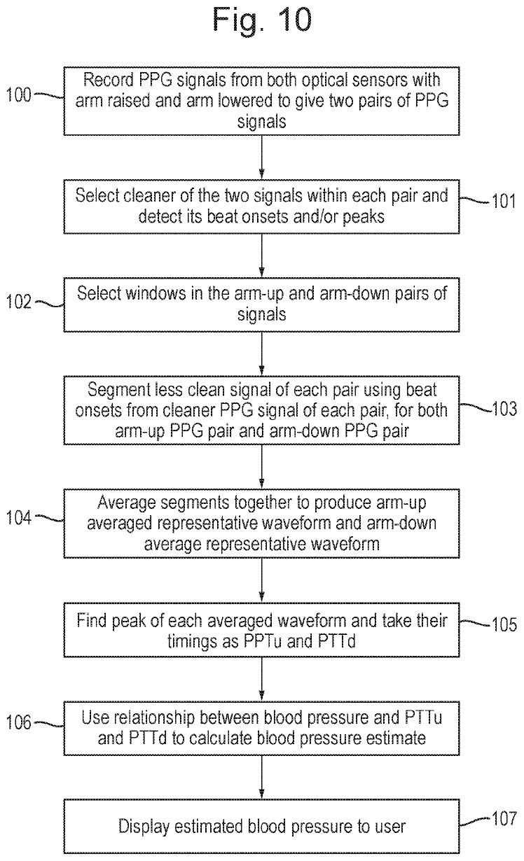

FIG. 10 is a flow diagram of an embodiment of the invention;

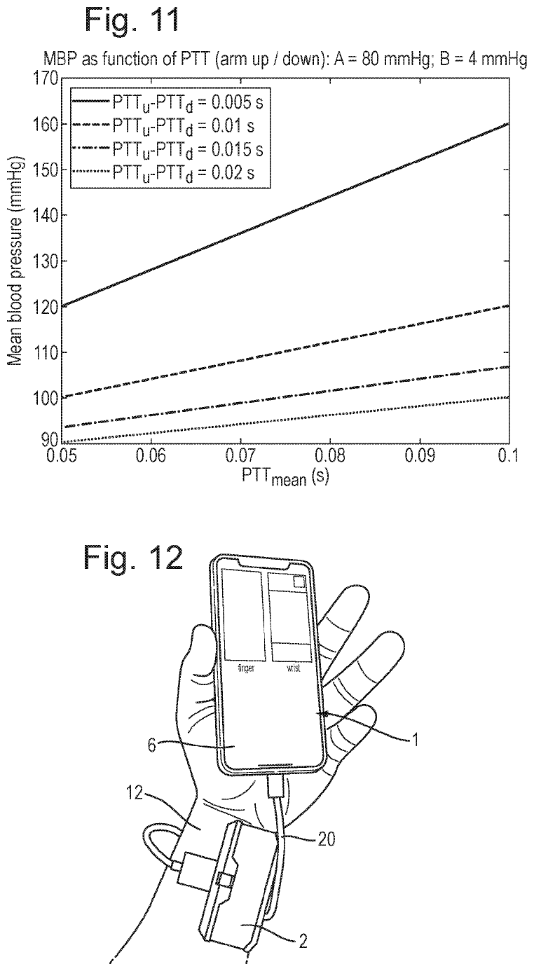

FIG. 11 is a calibration plot for a subject for the first embodiment of the invention;

FIG. 12 illustrates a second embodiment of the invention;

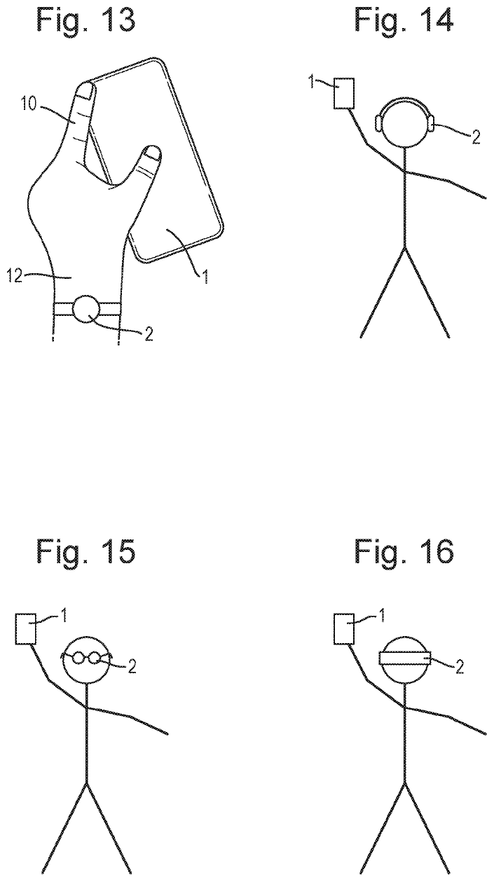

FIG. 13 schematically shows a third embodiment of the invention;

FIG. 14 schematically shows a fourth embodiment of the invention;

FIG. 15 schematically shows a fifth embodiment of the invention; and

FIG. 16 schematically shows a sixth embodiment of the invention.

FIG. 1 illustrates an embodiment of the invention in which the first device 1 is a mobile telephone (e.g. smartphone) with an attached accessory camera as the second device 2. In this embodiment the attached accessory camera is a thermal imaging camera including an IR sensor as the second optical sensor 8, though a conventional visible light camera may be used as an alternative 9. The mobile telephone 1 includes a built-in camera 3 as the first optical sensor, which includes a light source 4 (which can be used as the camera flash or for general illumination) and a light sensor 5 which can conventionally operate as a still camera or video camera as is conventional in modern smartphones. It preferably also includes, as is conventional, accelerometers allowing it to detect its own orientation and a touchscreen constituting both a display 6 and user-input device 7.

In a second embodiment of the invention illustrated in FIG. 12 the thermal camera 2 is not directly attached to the mobile telephone 1, but rather connected through a data communication cable 20 and held to the wrist in the manner of any wrist-worn device. The data cable can be replaced by a wireless communication link such as Bluetooth, wifi, etc.

As an alternative second device 2 to the attached accessory camera, alternative embodiments of the invention may use an optical sensor fitted to any convenient device which can be held in contact with the subject's skin, such a wristwatch, a conventional pulse oximeter, headphones or earphones, headset, spectacles etc. The second device 2 includes a light source and light detector which together form the second optical sensor 8, so that it can detect the reflectance PPG signal, and is in data communication with the first device 1 so that it can be controlled to perform the PPG measurement and can return the PPG signal to the first device 1 for processing.

FIGS. 2 and 3 are schematic illustrations of the embodiment of FIG. 1 in use in a blood pressure measurement. As can be seen the mobile telephone 1 is held by the user with the user's finger 10 in contact with the camera 3 and the attached accessory camera 2 facing the user's wrist 12. The second device 2 need not be in direct contact with the subject's skin, but is close-enough that it can detect the PPG signal in the blood vessels of the user's wrist.

In this embodiment the measurement protocol involves making a first measurement with the arm in a natural lowered position as illustrated schematically in FIG. 4A and a second measurement with the arm vertically raised as schematically illustrated in FIG. 4B. In this embodiment the process is under the control of an executable software application on the mobile telephone which conveniently instructs the user in the protocol, and may also show animation to the user directing them how to take the first and second measurement with the arm positioned downwardly and upwardly. The first device 1 may utilise in-built accelerometers or orientation detectors to check that the user takes the two measurements with the arm in the correct positions, and can thus automatically detect when the two measurements have been completed, enabling it to automatically start processing the measurements to calculate the blood pressure. Alternatively, the user can indicate completion of the protocol by input (clicking an appropriate completion button on a touchscreen of the device 1).

FIG. 5a illustrates a two minute segment of a PPG signal and its frequency content (FFT) measured in the index finger of a user pressed against the camera 3 as illustrated in FIG. 2. The FFT spectral power plot showing a clear heart rate signal at about 1.26 Hz corresponding approximately to a heart rate of 76 beats per minute. FIG. 5b shows a two minute segment of the PPG signal and its frequency content measured from the wrist of a user using a thermal imaging camera 2 attached to a mobile telephone 1 as illustrated in FIGS. 2 and 3. The spectral power plot of the signal shows that the signal is less clean than that obtained from the finger in contact with the mobile telephone 1, but nevertheless the heart rate signal is clearly present at 1.26 Hz, albeit with other frequency content related to noise and movement. In both of the time plots the left-hand half, approximately the first minute, was performed with the user's arm vertically down, and in this case the signal from the index finger is much stronger than the signal from the wrist. The right-hand half, the second minute, is the signal with the arm in the raised position, and for this period the signal from the wrist is stronger than before.

FIG. 10 is a flow diagram explaining the measurement protocol and the data processing. In step 100 both optical sensors 3 and 8 are used to record two PPG signals, with the arm of the subject raised and with the arm of the subject lowered. The aim of the method will be to detect the relative timings of the two PPG signals and to calculate from this the blood pressure. To improve the accuracy of the measurement, signal averaging is used to produce a representative averaged PPG waveform.

Therefore, in step 101 whichever of the two PPG signals is cleaner (i.e. has a higher signal-to-noise ratio) is taken and the maxima (beat peaks) or minima (beat onsets) are detected. FIGS. 6a and b illustrate the signals from FIGS. 5a and b with the beat peaks and onsets in the PPG signal originating from the finger marked with dots. These peaks or beat onsets will be used as reference timing points for the signal averaging.

In step 102 a window of the two signals in which the arm is pointed down is selected (the window may be from 4-15 seconds long and is selected such that the two signals (finger PPG and wrist PPG) are of good quality and there is not significant hand motion), and in step 103 the second (less clean) PPG signal window is segmented at the reference timing points, e.g. the beat onsets, from the first (cleaner) signal. Then in step 104 each of the segments of the second PPG signal are averaged together resulting in a single representative “arm-down” average waveform for that window. FIGS. 7a and b illustrate examples of magnified 15-second windows of the finger and wrist PPG signals from the “arm down” period of FIG. 6, with the beat onsets and beat peaks illustrated again with dots. The signal of FIG. 7b is segmented at the beat onset timings from FIG. 7a and the segments are averaged together to produce the representative “arm-down” average waveform.

Steps 102 to 104 are also performed on a period of the PPG signals in which the arm is pointed up, as shown in FIGS. 8a and 8b, and the waveforms in the second PPG signal are averaged by segmenting the second PPG signal of FIG. 8b at the timings of the beat onsets of the first PPG signal illustrated in FIG. 8a to produce a single representative “arm-up” average waveform for the second PPG signal.

FIGS. 9a and b shows the representative averaged waveforms for each of the two PPG signals in each of the two positions resulting from the signals shown in of FIGS. 7 and 8. It can be seen that there is a relative timing difference between them of about 0.2 seconds resulting from the different effect of gravity on the pulse transit time with the arm up and down.

In step 105 the relative timing of the arm-down and arm-up PPG signals is taken, for example the timing of their peaks with respect to the t=0 line (the y-axis of FIGS. 9a and 9b), as two pulse transit times PTT_{d} and PTTᵤ.

Then in step 106 the two pulse transit times are used together with a previously obtained relationship between the blood pressure and pulse transit time for this subject to calculate an estimate of the blood pressure of the subject. In step 107 the calculated estimate of blood pressure is displayed to the user on the screen of the mobile telephone 1.

The relationship between the two pulse transit times PTT_{d} and PTTᵤ and blood pressure p is of the form:

$$p = A + B ( \frac{P T {T}_{u}^{2} + P T {T}_{d}^{2}}{P T {T}_{u}^{2} - P T {T}_{d}^{2}} )$$

Where A and B are constants for each individual. A and B are obtained by a calibration process comprising measuring the blood pressure of the subject using any of the conventional methods e.g. an inflatable cuff, while also measuring the pulse transit times PTT_{d} and PTTᵤ with the arm raised and arm lowered using the device and method of the embodiment above. This is performed over a range of different blood pressures (at least two, but preferably more) which can be induced by having the user perform the measurements at different times of day (blood pressure varies naturally through the day, typically peaking in the morning and evening and dropping in the early afternoon and night), or while having the subject assume different postures, e.g. standing, seated and lying. FIG. 11 illustrates a calibration plot of mean blood pressure as a function of mean pulse transit time (PTT_{d}+PTTᵤ)/2 for different arm up/arm down PTT differences.

Although the invention is not limited to any particular theoretical basis for the relationship between pulse wave velocity and blood pressure as it is based on a measured calibration process as discussed above, the basis for the relationship used above may be derived by starting with the published result for pulse wave velocity derived by S. J. Payne in the paper “Analysis of the effects of gravity and wall thickness in a model of blood flow through axisymmetric vessels”, Med. Biol. Eng. Comput., 2004, 42, 799-806:

$${c}^{2} = \frac{β {G}_{0}}{2 ρ} {( \frac{A}{{A}_{0}} )}^{β / 2} - \frac{{h}_{0}}{{R}_{0}} ( \frac{{A}_{0}}{A} ) \frac{{p}_{e x t}}{ρ} \qquad ( 1 )$$

where the symbols have their standard meaning as defined in Payne (2004). Note that this is the simplified version of Equation 32 in Payne (2004), where it is assumed that the wave speed is much larger than the mean velocity of the flow.

The pressure-area relationship given by Payne (2004) is then used:

$$p - {p}_{0} = {P}_{ext} ( 1 + \frac{{h}_{0}}{{R}_{0}} \frac{{A}_{0}}{A} ) + {G}_{0} [ {( \frac{A}{{A}_{0}} )}^{β / 2} - 1 ] \qquad ( 2 )$$

noting that baseline conditions are denoted by the subscript 0. This is the generalised form of Equation 15 in Payne (2004), where an offset is added in the equation above to account for the fact that we are interested in absolute pressure.

As a first approximation, taking the external pressure to be negligible; Equation 2 can be substituted into Equation 1 to give:

$${c}^{2} ≅ \frac{β {G}_{0}}{2 ρ} ( p - {p}_{0} + {G}_{0} ) \qquad ( 3 )$$

Using this result, the two separate PTT tests are considered, one with arm up (subscript u) and one with arm down (subscript d). The average blood pressure along the arm will be altered due to gravity:

$$\begin{matrix}{p}_{u} = p - \frac{ρ g {z}_{u}}{2} & ( 4 ) \\ {p}_{d} = p + \frac{ρ g {z}_{d}}{2} & ( 5 )\end{matrix}$$

where the changes in height are given by zᵤ and z_{d} respectively. Hence three values of wave speed:

$$\begin{matrix}{c}_{0}^{2} = \frac{β {G}_{0}}{2 ρ} ( p - {p}_{0} + {G}_{0} ) & ( 6 ) \\ {c}_{u}^{2} = \frac{β {G}_{0}}{2 ρ} ( p - \frac{ρ {gz}_{u}}{2} - {p}_{0} + {G}_{0} ) & ( 7 ) \\ {c}_{d}^{2} = \frac{β {G}_{0}}{2 ρ} ( p + \frac{ρ {gz}_{d}}{2} - {p}_{0} + {G}_{0} ) & ( 8 )\end{matrix}$$

Re-arranging gives:

$$p = {p}_{off } + \frac{ρ g ( {z}_{d} + {z}_{u} )}{2} ( \frac{{c}_{d}^{2} + {c}_{u}^{2}}{{c}_{d}^{2} - {c}_{u}^{2}} ) \qquad ( 9 )$$

i.e. based on half the difference in height between the two elevations (chosen as being most robust).

Note that the relationship in Equation 9 does still require one variable to be calibrated (termed here as offset pressure, p_{off}) but does remove the need for two variables to be calibrated.

Equation 9 can also be re-written in more general terms:

$$\begin{matrix}p = A + B ( \frac{{c}_{d}^{2} + {c}_{u}^{2}}{{c}_{d}^{2} - {c}_{u}^{2}} ) \\ where \text{:} & ( 10 ) \\ B = \frac{ρ g ( {z}_{d} + {z}_{u} )}{2} & ( 11 )\end{matrix}$$

In terms of pulse transit times, since the lengths are invariant, Equations 9 and 10 become straightforwardly:

$$\begin{matrix}p = {p}_{off } + \frac{ρ g ( {z}_{d} + {z}_{u} )}{2} ( \frac{P T {T}_{u}^{2} + P T {T}_{d}^{2}}{P T {T}_{u}^{2} - P T {T}_{d}^{2}} ) & ( 12 ) \\ p = A + B ( \frac{P T {T}_{u}^{2} + P T {T}_{d}^{2}}{P T {T}_{u}^{2} - P T {T}_{d}^{2}} ) & ( 13 )\end{matrix}$$

noting that the bracketed term is non-dimensional and thus any convenient units can be used for PTT.

FIGS. 13 to 16 schematically illustrate alternative embodiments of the invention. In FIG. 13 the second optical sensor is in a wrist-worn device 2 and thus is in contact with the skin of the subject. In FIG. 14 the second optical sensor is in an ear piece worn on the head of the user and is thus close to or in contact with skin of the ear. In FIG. 15 the second optical sensor is in spectacles worn on the head of the user and in FIG. 16 the second optical sensor is in a VR headset worn on the head of the user.

1. A method of estimating blood pressure comprising:
detecting a first photoplethysmographic signal at a first location on a subject's body using a first optical sensor of a first device held by the subject's hand;
detecting a second photoplethysmographic signal at a second location on the subject's body using a second optical sensor of a second device;
measuring the relative timings of the detected photoplethysmographic signals and calculating from the relative timings an estimate of the blood pressure.

2. A method according to claim 1 further comprising detecting the first and second photoplethysmographic signals with the subject's arm in a first position and measuring a first relative timing between the detected photoplethysmographic signals, detecting the first and second photoplethysmographic signals with the subject's arm in a second position, different from the first position, and measuring a second relative timing between the detected photoplethysmographic signals, calculating from the first and second relative timings an estimate of the subject's blood pressure.

3. A method according to claim 2 wherein the first position is with the arm raised higher than in the second position.

4. A method according to claim 1 wherein the first location is on the hand of the subject, for example on the finger.

5. A method according to claim 1 wherein the second location is one of: the wrist of the subject, on the head of the subject, on or adjacent to the ear of the subject.

6. A method according to claim 1 wherein at least one of the optical sensors is placed in contact with subject's skin to detect the photoplethysmographic signal.

7. A method according to claim 1 wherein at least one of the optical sensors is an optical reflectance measuring device.

8. A method according to claim 1 wherein at least one of the optical sensors comprises a light source for emitting light towards the subject's skin and a light detector for detecting light emitted from the subject's skin.

9. A method according to claim 1 wherein the first device is a mobile computing device incorporating a camera as said first optical sensor and the second device is one of: an accessory camera as said second optical sensor, a wrist-worn device, a head-worn device, an audio headphone, a headset, spectacles, each comprising an optical sensor as said second optical sensor.

10. A method according to claim 1 further comprising one of the steps of (a) synchronizing clocks of the first and second devices, or (b) acquiring the time difference between clocks of the first and second devices.

11. A method according to claim 1 comprising the steps of averaging together a plurality of photoplethysmographic waveforms of said photoplethysmographic signals to obtain representative average photoplethysmographic waveforms and measuring said relative timing using the representative average photoplethysmographic waveforms.

12. A method according to claim 11 further comprising the step of comparing the signal-to-noise ratios of the first and second photoplethysmographic signals, selecting the photoplethysmographic signal with the highest signal-to-noise ratio and detecting its amplitude maxima or minima; using the timings of the detected maxima or minima as reference timings for the averaging of the plurality of photoplethysmographic waveforms.

13. A method according to claim 1 wherein the first and second devices are connected together for data communication.

14. A method according to claim 1 wherein the first device controls the second device and performs said relative timing measurement.

15. A method according to claim 1 wherein the second device is physically mounted to and electrically connected to the first device.

16. A method of calculating an estimate of blood pressure from a measurement of pulse transit time comprising the steps of: measuring a first pulse transit time in a subject's arm with the subject's arm in a first position; measuring a second pulse transit time in a subject's arm with the subject's arm in a second position, different from the first position, and measuring the difference between the first and second pulse transit times, and calculating from the difference in first and second pulse transit times an estimate of the subject's arterial blood pressure.

17. A method according to claim 16 wherein the first position is with the arm raised higher than in the second position.

18. A method according to claim 16 wherein the pulse transit times are measured between a first location on the hand of the subject, for example on the finger, and a second location elsewhere on the subject.

19. A method according to claim 18 wherein the second location is one of: on the wrist of the subject, on the head of the subject, on or adjacent to the ear of the subject.

20. Apparatus for measuring blood pressure comprising: a first device comprising a first optical sensor for detecting a first photoplethysmographic signal at a first location on a subject's body, the first device being a handheld device; a second device comprising a second optical sensor for detecting a second photoplethysmographic signal at a second location on the subject's body; a programmable data processor configured to control the first and second devices to execute the method of claim 1.

21. Apparatus according to claim 20 wherein the first device is a mobile computing and communications device, the first optical sensor is a camera incorporated into the first device, and the programmable data processor is a data processor of the first device.

22. Apparatus according to claim 21 wherein the mobile computing and communications device is one of a smartphone or a tablet computer.

23. Apparatus according to claim 20 wherein the second device is one of: an accessory camera as said second optical sensor, a wrist-worn device, a head-worn device, an audio headphone, spectacles, each comprising an optical sensor as said second optical sensor.

24. Apparatus according to claim 20 wherein the second device is connected for data communication with said first device.

25. Apparatus according to claim 20 wherein said second device is physically mounted to said first device.
